(19) United States
(12) Patent Application Publication
(10) Pub. No.: US 2023/0037015 A1
(43) Pub. Date: Feb. 2, 2023

(54) Material Development Support Apparatus, Material Development Support Method, and Material Development Support Program

(71) Applicant: Nippon Telegraph and Telephone Corporation, Tokyo (JP)

(72) Inventors: Kenta Fukada, Tokyo (JP); Michiko Seyama, Tokyo (JP)

(21) Appl. No.: 17/784,909

(22) PCT Filed: Dec. 16, 2019
(86) PCT No.: PCT/JP2019/049168

§ 371 Date: Jun. 13, 2022

(51) Int. Cl. G16C 60/00; G06N 3/04; G16C 20/30; G16C 20/70

(52) U.S. Cl. CPC G16C 60/00; G06N 3/0454; G16C 20/30; G16C 20/70

(57) ABSTRACT

An embodiment includes a materials development support apparatus including an input data acquisition device configured to acquire input data including a material of a base forming a thin film and a function of the thin film, a candidate data generator configured to provide a preset verification target material as an input to a first learning, output a plurality of candidates for a function provided by the verification target material, an inverse analyzer configured to select a material that provides the function of the thin film included in the input data from the plurality of candidates for the function included in the candidate data, provide the material of the base included in the input data and the selected material as inputs to a second learning model, output a candidate for structure of the thin film, and a presenter configured to present the candidate for the structure of the thin film output.

### CROSS-REFERENCE TO RELATED APPLICATIONS

This application is a national phase entry of PCT Application No. PCT/JP2019/049168, filed on Dec. 16, 2019, which application is hereby incorporated herein by reference.

### TECHNICAL FIELD

The present invention relates to a materials development support apparatus, a materials development support method, and a materials development support program.

### BACKGROUND

In recent years, data-driven materials development using information science and computational science methods called materials informatics has made remarkable progress. Materials informatics has attracted a great deal of attention as a comprehensive and rapid materials search technique that cannot be easily performed by conventional experimental methods.

The fields covered by materials informatics are diverse, for example, batteries, catalysts, and biomaterials. Furthermore, there have been studied various approaches such as materials design technology using computational science at the atomic and molecular level such as molecular dynamics simulation and exploration of synthetic routes and optimization in combination with artificial intelligence (AI) technology such as machine learning.

In the field of such conventional materials informatics, there are many cases where a target whose properties can be expressed by energy calculation is selected, mainly for thermoelectric conversion, conductivity, catalytic activity, binding of a ligand and a receptor, and the like.

However, when it is difficult to have a mathematically unified discussion, for example, when “multiple functions” such as biocompatibility, machine durability, and transparency are targeted, there may be a case difficult to handle since the functions may have a trade-off relationship or may be independent from each other. Consequently, there are still only a small number of cases where materials informatics is applied if multiple functions are targeted.

However, in order to bring the product into practical use, it is demanded that not only one function but a plurality of functions achieve performance at a certain level or higher at the same time, in consideration of safety, durability, price, and the like. Therefore, it can be said that it is also important to realize a materials development technique targeting a plurality of functions in the field of materials informatics.

For example, Non Patent Literature 1 discloses a technique for performing data-driven thin film designing that achieves multiple functions by using text information such as papers in the past as learning data. In Non Patent Literature 1, based on several hundreds of papers on “thin film”, chemical properties such as a functional group of a monomolecular film as input information and multiple functions such as a contact angle and b100d adhesion performance as output information are learned as correct answer labels. Non Patent Literature 1 facilitates the data-driven development of thin films based on this learning data.

### CITATION LIST

### Non Patent Literature

[NPL 1] Hiroyuki Tahara et al. “Data-driven Design of Protein- and Cell-resistant Surfaces: A Challenge to Design Biomaterials Using Material Informatics” Vacuum and Surface Vol.62, No. 3 (Mar. 10, 2019):pp.141-146.

### SUMMARY

### Technical Problem

Prior arts focus on an absorption phenomenon at an interface between a biomolecule and a monomolecular film by using a “monomolecular film” having multiple functions. However, the monomolecular film has an issue of durability, and there is an issue that the same method cannot be applied to a “multi-layer film” having multiple interfacial surfaces.

In addition, to create learning data, elements, functional groups, bonds, etc. in the film need to be manually read out from the data in the paper. Such a high hurdle for constructing database has also been an issue. In particular, in designing a multi-layer film, processing for determining whether another layer can be formed on top of one layer and a method for constructing databased by using data mechanically collected from the text in the papers are newly needed. On this account, with the technique described in NPL 1, it has been difficult to expand a target of materials informatics to a “multi-layer film” and to further facilitate data collection.

The embodiments of the present invention has been made to solve the above problem, and an object of the embodiments of the present invention is to more easily present a candidate for the design of a multi-layer film having multiple functions. The embodiments of present invention relate to a materials development support apparatus, a materials development support method, a materials development support program, and a materials informatics technique.

### Means for Solving the Problem

To solve the above problem, a materials development support apparatus according to embodiments of the present invention includes: an input data acquisition unit that acquires input data including a material of a base forming a thin film and a function of the thin film; a candidate data generation unit that provides a preset verification target material as an input to a first learning model in which a relationship between an individual one of a plurality of materials used for forming a thin film and a function provided by the material is previously learned, performs an operation of the first learning model, outputs a plurality of candidates for a function provided by the verification target material, and generates candidate data; an inverse analysis unit that selects a material that provides the function of the thin film included in the input data from the plurality of candidates for the function included in the candidate data, provides the material of the base included in the input data and the selected material as inputs to a second learning model in which compatibility with the base forming the thin film is previously acquired by learning, performs an operation of the second learning model, and outputs a candidate for structure of the thin film; and a presentation unit that presents the candidate for the structure of the thin film output by the inverse analysis unit.

To solve the above problem, a materials development support apparatus according to embodiments of the present invention includes: a first extraction unit that extracts a plurality of preset function names indicating a function of a thin film from an individual one of a plurality of document data; a second extraction unit that extracts a plurality of preset material names indicating a material used for forming the thin film from an individual one of a plurality of document data; a first learning data generation unit that generates first learning data in which a material and a function provided by the material are associated with each other for each of the plurality of material names, based on the plurality of function names extracted by the first extraction unit and the plurality of material names extracted by the second extraction unit; a first learning data generation unit that generates second learning data in which the individual material indicated by the plurality of material names and compatibility with the base forming the thin film are associated with each other, based on the plurality of function names extracted by the first extraction unit, the plurality of material names extracted by the second extraction unit, and the extraction-source document data; a first learning processing unit that trains a preset first machine learning model by using the first learning data and constructs the first learning model in which a relationship between a material and a function provided by the material is learned; a second learning processing unit that trains a preset second machine learning model by using the second learning data and constructs the second learning model in which compatibility with the base forming the thin film is acquired by learning; a first learning model storage unit that stores the trained first learning model; a second learning model storage unit that stores the trained second learning model; and an output unit that transmits the first learning model and the second learning model to outside.

To solve the above problem, a materials development support method according to embodiments of the present invention includes: an input data acquisition process that acquires input data including a material of a base forming a thin film and a function of the thin film; a candidate data generation process that provides a preset verification target material as an input to a first learning model in which a relationship between an individual one of a plurality of materials used for forming a thin film and a function provided by the material is previously learned, performs an operation of the first learning model, outputs a plurality of candidates for a function provided by the verification target material, and generates candidate data; an inverse analysis process that selects a material that provides the function of the thin film included in the input data from the plurality of candidates for the function included in the candidate data, provides the material of the base included in the input data and the selected material as inputs to a second learning model in which compatibility with the base forming the thin film is previously acquired by learning, performs an operation of the second learning model, and outputs a candidate for structure of the thin film; and a presentation process that presents the candidate for the structure of the thin film output in the inverse analysis process.

To solve the above problem, a materials development support program that causes a computer to execute: an input data acquisition process that acquires input data including a material of a base forming a thin film and a function of the thin film; a candidate data generation process that provides a preset verification target material as an input to a first learning model in which a relationship between an individual one of a plurality of materials used for forming a thin film and a function provided by the material is previously learned, performs an operation of the first learning model, outputs a plurality of candidates for a function provided by the verification target material, and generates candidate data; an inverse analysis process that selects a material that provides the function of the thin film included in the input data from the plurality of candidates for the function included in the candidate data, provides the material of the base included in the input data and the selected material as inputs to a second learning model in which compatibility with the base forming the thin film is previously acquired by learning, performs an operation of the second learning model, and outputs a candidate for structure of the thin film; and a presentation process that presents the candidate for the structure of the thin film output in the inverse analysis process.

### Effects of the Invention

According to embodiments of the present invention, a material that provides a function of a thin film included in input data is selected from a plurality of candidates for a function included in the candidate data, and a material of a base included in the input data and the selected material are given as inputs to a second learning model in which compatibility with the base forming the thin film is previously acquired by learning. Next, an operation of the second learning model is performed, and a candidate for the structure of the thin film is output. In this way, the candidate for the design of the multi-layer film can be presented more easily.

### BRIEF DESCRIPTION OF THE DRAWINGS

FIG. 1 is a block diagram illustrating a functional configuration of a materials development support apparatus according to a first embodiment of the present invention.

FIG. 2 is a block diagram illustrating an example of a computer configuration that achieves the materials development support apparatus according to the first embodiment.

FIG. 3 is a block diagram illustrating an example of a specific configuration of a materials development support apparatus according to the present invention.

FIG. 4 is a diagram for describing a use example of the materials development support apparatus according to the present invention.

FIG. 5 is a flowchart for describing a materials development support method according to the first embodiment.

FIG. 6 is a diagram for describing extraction processing according to the first embodiment.

FIG. 7 is a flowchart for describing the extraction processing according to the first embodiment.

FIG. 8 is a diagram for describing learning data generation processing according to the first embodiment.

FIG. 9 is a flowchart for describing the learning data generation processing according to the first embodiment.

FIG. 10 is a diagram for describing learning processing according to the first embodiment.

FIG. 11 is a diagram for describing learning processing according to the first embodiment.

FIG. 12 is a block diagram illustrating a functional configuration of a materials development support apparatus according to a second embodiment.

FIG. 13 is a flowchart for describing a materials development support method according to the second embodiment.

FIG. 14 is a diagram for describing generation processing of candidate data according to the second embodiment.

FIG. 15 is a diagram for describing inverse analysis processing according to the second embodiment.

FIG. 16 is a flowchart for describing the inverse analysis processing according to the second embodiment.

FIG. 17 is a diagram for describing effects of the materials development support apparatus according to the second embodiment.

### DETAILED DESCRIPTION OF ILLUSTRATIVE EMBODIMENTS

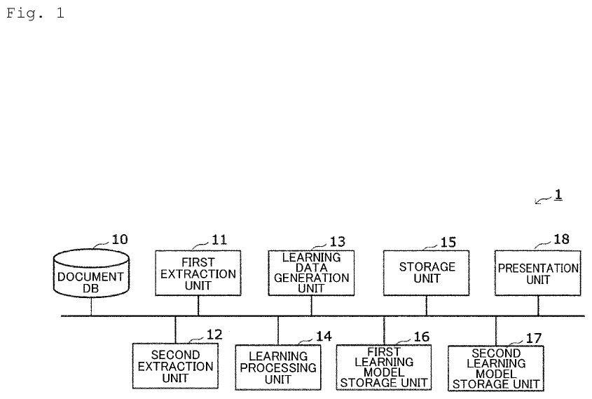

Hereinafter, a preferred embodiment of the present invention will be described in detail with reference to FIGS. 1 to 17.

Outline of Embodiments of the Invention

First, an outline of a materials development support apparatus 1 according to an embodiment of the present invention will be described. The materials development support apparatus 1 according to the present embodiment extracts preset function names indicating a function of a thin film and preset material names indicating a material used for forming the thin film from a plurality of document data such as papers and generates learning data used in machine learning based on the extracted data.

The materials development support apparatus 1 trains a machine learning model (a first machine learning model) prepared in advance based on the learning data and constructs a first learning model in which a relationship between a material and a function provided by the material is learned. In addition, the materials development support apparatus 1 trains a preset machine learning model (a second machine learning model) by using the learning data and constructs a second learning model in which compatibility with a base forming the thin film is acquired by learning. Further, the materials development support apparatus 1 outputs the first learning model and the second learning model that have been trained to the outside.

### First Embodiment

First, an outline of a configuration of the materials development support apparatus 1 according to a first embodiment of the present invention will be described. The materials development support apparatus 1 according to the first embodiment performs learning processing using machine learning and constructs a trained first learning model and a trained second learning model. FIG. 1 is a block diagram illustrating a functional configuration of the materials development support apparatus 1.

Functional Block of Materials Development Support Apparatus

The materials development support apparatus 1 includes a document DB 10, a first extraction unit 11, a second extraction unit 12, a learning data generation unit 13, a learning processing unit 14, a storage unit 15, a first learning model storage unit 16, a second learning model storage unit 17, and a presentation unit 18.

The document DB 10 stores text information such as papers. In the document DB 10, a plurality of documents related to a specific technique, for example, a thin film, is stored in advance. The document DB 10 can store document data in a specific language, for example, in English. For example, in a case of a paper, the document data stored in the document DB lo includes text data other than image data, such as titles, summaries, experimental methods, results, and consideration.

Hereinafter, a “sentence” means text data. Further, the “sentence” refers to text data of a character string divided by a punctuation mark or a period, and a “document” refers to a file of text data in a natural language including text composed of a plurality of “sentences”.

The first extraction unit 11 extracts a plurality of preset function names indicating a function of a thin film from an individual one of the plurality of document data stored in the document DB 10. In the present embodiment, the “function” includes, for example, not only a function that can be represented by energy calculation or the like in a mathematically uniform manner, such as thermoelectric conversion, but also information having relatively low mathematical relevance. For example, durability, transparency, liquid repellency, and flexibility can be listed as the function of the thin film. Words related to these preset functions are stored in the storage unit 15. For example, the first extraction unit 11 extracts a word indicating the function stored in the storage unit 15, such as “wettability” and “conductivity”, from the document data. In the present embodiment, the first extraction unit 11 can extract a word indicating the function from each of the document data sets.

The second extraction unit 12 extracts a plurality of preset material names indicating a material used for forming the thin film from an individual one of the plurality of document data stored in the document DB 10. The “material” includes, for example, a functional group such as “methyl”, “ethyl”, “vinyl”, and “fluoro”, a metal composition, and the material of a substrate (base) such as “glass” and “cellulose”. The second extraction unit 12 extracts words indicating the materials stored in the storage unit 15 from the document data. The second extraction unit 12 can extract the word indicating the material from each of the document data sets.

The first extraction unit 11 and the second extraction unit 12 can use a known character string search algorithm such as the Boyer-Moore (BM) algorithm and the Knuth-Morris-Pratt (KMP) algorithm when detecting a specific word from the document data. The extraction data including the “material” and the “function” extracted from each of the document data sets by the first extraction unit 11 and the second extraction unit 12 is stored in the storage unit 15.

The learning data generation unit 13 generates learning data based on the extraction data in which words indicating the preset “function” and “material” are extracted by the first extraction unit 11 and the second extraction unit 12.

More specifically, based on the plurality of function names extracted by the first extraction unit 11 and the plurality of material names extracted by the second extraction unit 12, the learning data generation unit (first learning data generation unit) 13 generates first learning data in which a material and a function provided by the material are associated with each other for each of the plurality of material names. Compatibility between the materials is a reference that reflects the material properties, which are taken into consideration when forming a thin film.

For example, among the materials used in the consecutive processes or the same process, the materials that have good compatibility in terms of the order of manufacturing a thin film and that have actually been used in similar procedures are defined as having good compatibility. In contrast, the materials that have poor compatibility in terms of the order of manufacturing a thin film and that have never been actually used in similar procedures are defined as having poor compatibility. There is a certain ordering in selecting film-forming materials, and information reflecting this ordering is the compatibility between the materials. The first learning data is, for example, data in which information indicating compatibility is added to a combination of two materials as a correct answer label.

The learning data generation unit 13 divides text data that is included in the document data and that indicates a plurality of consecutive processes related to the film-forming process into segments each constituting one process. Further, when a material A in the preceding stage and a material B in the subsequent stage appear in the same process or the consecutive processes, the learning data generation unit 13 adds a label indicating good compatibility to the material A and the material B. The consecutive processes refer only to a case where a layer is first formed with the material A in the preceding stage, and the next layer is formed with the material B in the subsequent stage. If a layer is first formed with the material B in the subsequent stage, and the next layer is formed with the material A in the preceding stage in the consecutive processes, these materials are not deemed to have good compatibility. For example, while it is common to have a glass substrate as the material in the preceding stage and an etching solution as the material in the subsequent stage, it is impossible to have an etching solution as the material in the preceding stage and a glass substrate as the material in the subsequent stage as the manufacturing order.

Further, the learning data generation unit (second learning data generation unit) 13 generates a second learning data in which the individual material indicated by the plurality of material names and compatibility with the base (substrate) forming the thin film are associated with each other, based on the plurality of function names extracted by the first extraction unit 11, the plurality of material names extracted by the second extraction unit 12, and extraction-source document data. For example, a conductive material is used for a heater film by Joule heat. Further, the same conductive material may be used as an electromagnetic shielding film. Each material contributes to achieving a function in accordance with an intended use.

As described above, the second learning data is data in which the function of each material extracted by the first extraction unit 11 is added to the material extracted by the second extraction unit 12 as a correct answer label. The first learning data and the second learning data generated by the learning data generation unit 13 are stored in the storage unit 15.

The learning processing unit 14 trains a learning model such as a machine learning model prepared in advance by using the learning data generated by the learning data generation unit 13 and constructs a trained model. For example, the learning processing unit 14 can perform supervised learning on a known machine learning model such as a multi-layer neural network including a recurrent neural network (RNN), an autoencoder, a convolutional neural network (CNN), and an LSTM network. Alternatively, the machine learning model to be trained can be set as desired, and not only supervised learning but also semi-supervised learning or the like can also be adopted.

More specifically, the learning processing unit (first learning processing unit) 14 trains a preset machine learning model using the first learning data and constructs a first learning model in which a relationship between a material and a function provided by the material is learned. For example, the learning processing unit 14 trains the multi-layer neural network to update and adjust a feature amount representing the compatibility between two materials, that is, a value of the configuration parameter of the multi-layer neural network and determines a final value. The first learning model constructed by the learning using the first learning data is stored in the first learning model storage unit 16.

Further, the learning processing unit (second learning processing unit) 14 trains a preset machine learning model using the second learning data and constructs a second learning model in which compatibility with the base forming the thin film is acquired by the learning.

The storage unit 15 stores the extraction data including the functions and materials of the thin film extracted from the document data by the first extraction unit 11 and the second extraction unit 12. In addition, the storage unit 15 stores the first learning data and the second learning data generated by the learning data generation unit 13. Further, the storage unit 15 stores information about preset machine learning models used by the learning processing unit 14 as learning targets.

The first learning model storage unit 16 stores the trained first learning model constructed by the learning processing unit 14. More specifically, the first learning model storage unit 16 stores values of weight parameters of the multi-layer neural network determined in the learning processing by the learning processing unit 14, etc.

The second learning model storage unit 17 stores the trained second learning model constructed by the learning processing unit 14.

The presentation unit (output unit) 18 can present the extraction data indicating the “material” and the “function” extracted from each of the document data sets by the first extraction unit 11 and the second extraction unit 12 and the trained first learning model and second learning model obtained in the learning processing by the learning processing unit 14 to an external server (not illustrated) or the like.

Hardware Configuration of Materials Development Support Apparatus

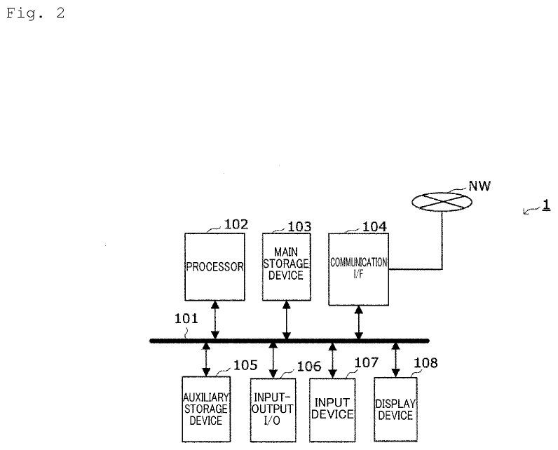

Next, an example of a computer configuration that implements the materials development support apparatus 1 having the above-described functions will be described with reference to FIG. 2.

As illustrated in FIG. 2, the materials development support apparatus 1 can be implemented, for example, by a computer including a processor 102, a main storage device 103, a communication I/F 104, an auxiliary storage device 105, an input-output I/O 106 connected via a bus 101 and a program that controls these hardware resources. For example, an input device 107 and a display device 108 provided outside are each connected to the materials development support apparatus 1 via the bus 101.

A program for causing the processor 102 to perform various controls and calculations is stored in the main storage device 103 in advance. The processor 102 and the main storage device 103 implement each function of the materials development support apparatus 1 including the first extraction unit ii, the second extraction unit 12, the learning data generation unit 13, and the learning processing unit 14 illustrated in FIG. 1.

The communication I/F 104 is an interface circuit for performing communication with various external electronic devices via a communication network NW.

As the communication I/F 104, for example, a communication control circuit and an antenna corresponding to wireless data communication standards such as 3G, 4G, 5G, a wireless LAN, and Bluetooth (registered trademark) are used.

The auxiliary storage device 105 is composed of a readable and writable storage medium and a drive device for writing and reading various kinds of information such as programs and data to and from the storage medium. A semiconductor memory such as a hard disk or a flash memory can be used as the storage medium of the auxiliary storage device 105.

The auxiliary storage device 105 has a program storage area for storing programs for causing the materials development support apparatus 1 to perform material development support processing including extraction processing, learning data generation processing, and learning processing. The auxiliary storage device 105 implements the storage unit 15, the first learning model storage unit 16, and the second learning model storage unit 17 described with reference to FIG. 1. The auxiliary storage device 105 may have, for example, a backup area for backing up the above-mentioned data, programs, and the like.

The input-output I/O 106 is composed of I/O terminals that input a signal from the external device and output a signal to the external device.

The input device 107 is composed of a keyboard, a touch panel, or the like, receives an operation input from the outside, and generates a signal corresponding to the operation input.

The display device 108 is implemented by a liquid crystal display or the like.

Example of Specific Configuration of Materials Development Support Apparatus

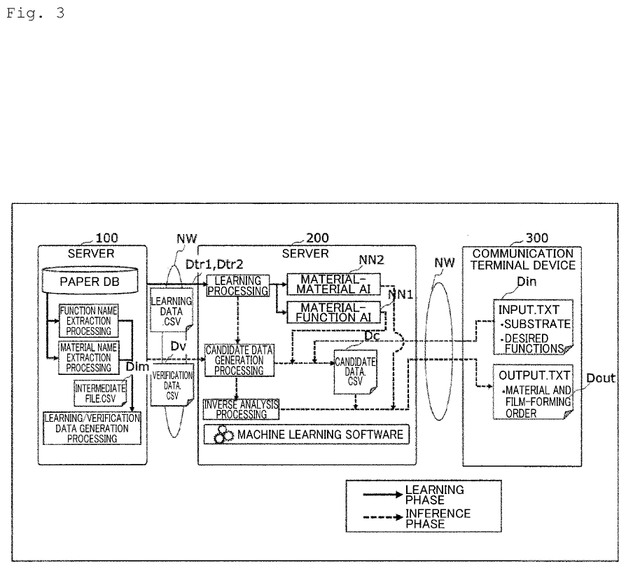

An example of a specific configuration of the materials development support apparatus 1 having the above-described configuration will be described with reference to a block diagram in FIG. 3. For example, the materials development support apparatus 1 can be implemented by servers 100, 200, and a communication terminal device 300. The servers 100, 200, and the communication terminal device 300 are connected via a communication network NW. A flow indicated by a solid line in FIG. 3 is a processing flow of the materials development support apparatus 1 according to the present embodiment (“learning phase” in FIG. 3). Thus, the materials development support apparatus 1 according to the first embodiment is implemented by the servers 100 and 200 involved in the learning phase.

The server 100 includes, for example, the document DB 10, the first extraction unit ii, the second extraction unit 12, and the learning data generation unit 13 described with reference to FIG. 1.

The server 200 includes, for example, the learning processing unit 14, the first learning model storage unit 16, and the second learning model storage unit 17 described with reference to FIG. 1.

The servers 100 and 200 are implemented by a computer configuration including a processor, a main storage device, a communication I/F, and an auxiliary storage device as described with reference to FIG. 2. Further, as illustrated in FIG. 3, the server 100 transmits generated learning data to the server 200 via the communication network NW.

As described above, the materials development support apparatus 1 according to the present embodiment can be implemented by the configuration in which each function illustrated in FIG. 1 is distributed on the network.

Materials Development Support Method

Next, an operation performed by the materials development support apparatus 1 having the above-described configuration will be described with reference to FIGS. 3 to 11.

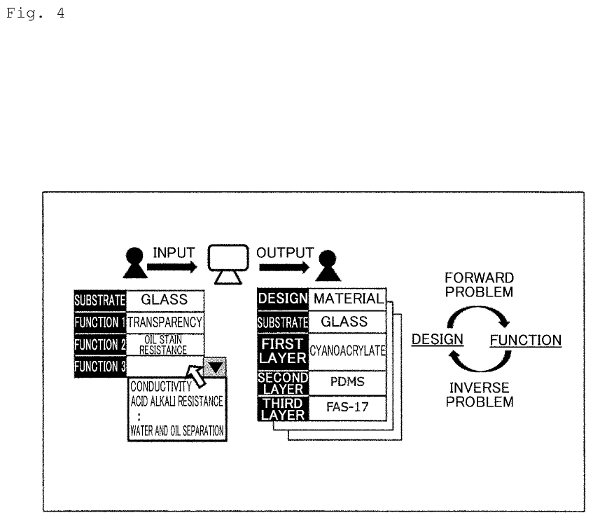

The materials development support apparatus 1 according to the present embodiment trains individually two machine learning models such as multi-layer neural network and constructs a trained first learning model and a trained second learning model. As illustrated in FIG. 4, the two learning models constructed by the materials development support apparatus 1 are used in inference processing, which will be described below. That is, by providing the material of a substrate used for a multi-layer film and a desired function of the multi-layer film specified by the user to the trained models as inputs, a candidate for the material of each layer of the multi-layer film is presented as an output.

Outline of Materials Development Support Method

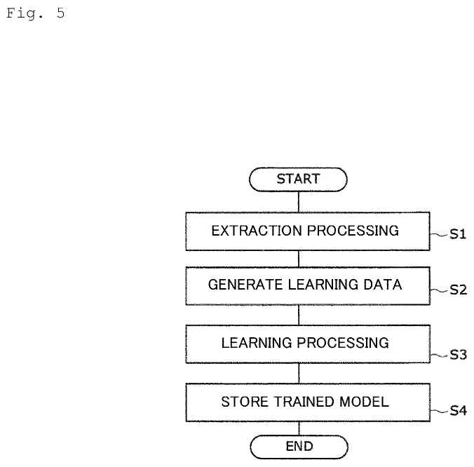

First, an outline of the operation performed by the materials development support apparatus 1 according to the present embodiment will be described with reference to a flowchart in FIG. 5.

As illustrated in FIG. 5, first, the first extraction unit 11 and the second extraction unit 12 extract words indicating preset “materials” and “functions” of a thin film from each of the document data sets stored in the document DB 10 (step S1).

Next, the learning data generation unit 13 generates first learning data indicating the function provided by the material and second learning data indicating the compatibility between two materials based on the words indicating the “materials” and the “functions” extracted in step S1 and the extraction-target document data (step S2).

Next, the learning processing unit 14 trains a predetermined machine learning model using the first learning data generated in step S2 and outputs a trained first learning model, and the learning processing unit 14 also trains a predetermined machine learning model using the second learning data and outputs a trained second learning model (step S3). More specifically, the learning processing unit 14 constructs a first learning model in which the compatibility between the materials is learned and a second learning model in which the relationship between the material and the function is learned.

Next, the trained first learning model and the trained second learning model are stored in the first learning model storage unit 16 and the second learning model storage unit 17, respectively (step S4).

Extraction Processing

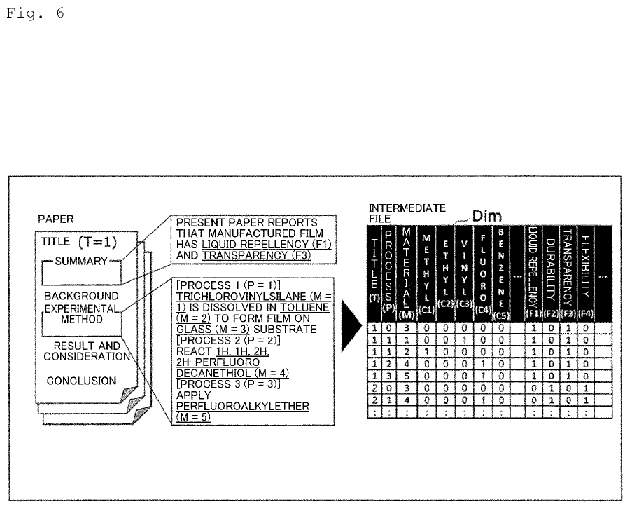

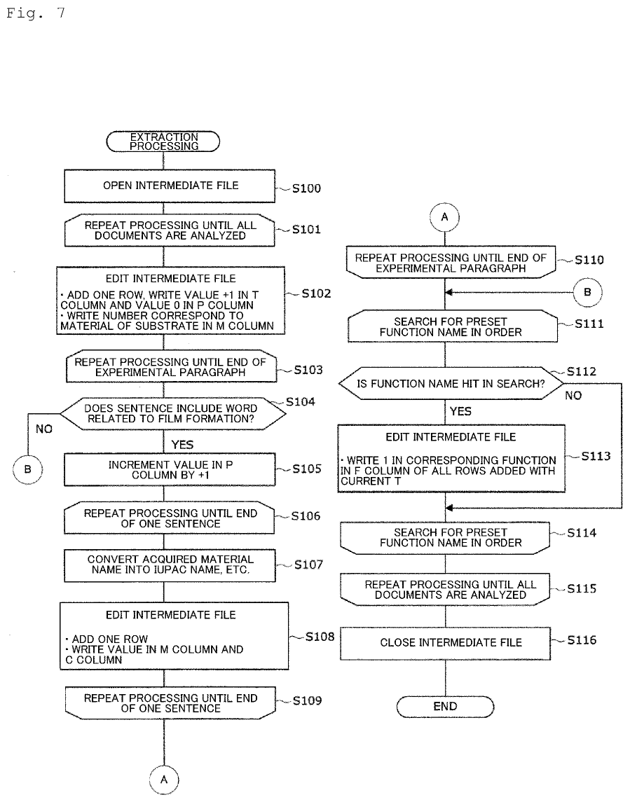

Next, a specific example of extraction processing performed by the first extraction unit 11 and the second extraction unit 12 will be described with reference to FIGS. 6 and 7. The following description will be made assuming that the document data stored in the document DB 10 is a plurality of papers related to a thin film.

As illustrated in FIG. 6, an intermediate file is created with the extraction data which is extracted by the first extraction unit 11 and the second extraction unit 12 and in which the material names including raw materials used in the film-forming process are extracted. For example, a text file in CSV format can be used as the intermediate file.

As illustrated in FIG. 6, a sentence including a word related to film formation such as “coated”, “sprayed”, and “modified” is defined as one process ([process 1 (P=1)] illustrated in FIG. 6, or the like). Further, the end of the sentence is determined by the appearance of a character representing a delimiter such as “period, comma, and then”. However, the end of the sentence can be freely defined.

Since a plurality of processes are performed when a multi-layer film is formed, the second extraction unit 12 extracts a material name used in each process and creates the extraction data in the intermediate file. The second extraction unit 12 performs the extraction processing on a paragraph of “experimental method” or the like included in paper data.

The first extraction unit 11 extracts a word related to a preset function, for example, “wettability”, “conductivity”, and the like (“liquid repellency (F1)”, “transparency (F3)”, etc. illustrated in FIG. 6), from a paragraph of “summary” or the like included in paper data. The intermediate file (extraction data) illustrated in FIG. 6 is created from the data extracted by the first extraction unit 11 and the second extraction unit 12.

Hereinafter, the extraction processing performed by the first extraction unit 11 and the second extraction unit 12 and implemented by the processor 102 will be described with reference to a flowchart illustrated in FIG. 7.

First, the processor 102 opens the intermediate file in which the extraction results are recorded (step S100). Next, the processor 102 starts 100p processing in which the processing from step S102 to step S113 are repeatedly performed on all of the plurality of paper data stored in the document DB 10 (step S10i).

Next, the processor 102 acquires one of the paper data sets from the document DB 10 and edits the intermediate file opened in step S100 (step S102). More specifically, as illustrated in “intermediate file Dim” in FIG. 6, the processor 102 adds one row to the intermediate file for each acquisition of the paper data set and sets a value in T column given to each “title” of the paper to +1 and a value in P column indicating “process” to 0. Further, the processor 102 identifies the material of a substrate from the entire paper data set and writes a corresponding material number as a value in M column indicating the “material” in the intermediate file.

Next, the processor 102 identifies a paragraph related to an experiment included in the paper data and repeatedly performs the processing from step S104 to step S109 on each sentence from the first to the last in the paragraph (step S103). For example, information that can identify the paragraph of “experimental method” and the paragraph of “summary” is previously given to the corresponding paragraph in each of the paper data sets stored in the document DB 10.

Next, the processor 102 identifies the paragraph of the experiment included in the paper data and extracts a sentence related to film formation (step S104). For example, the processor 102 performs the extraction in order from the first sentence of the paragraph of “experimental method” included in the paper data.

If the extraction target sentence includes a preset word related to film formation (step S104: YES), the processor 102 increments (+1) the value of the P column in the intermediate file (step S105). In contrast, if the extraction target sentence does not include a preset word related to film formation (step S104: NO), the processing proceeds to step S111 via connector B.

Next, the processor 102 repeatedly performs the processing in step S107 and step S108 until the end of one extraction target sentence (step S106). More specifically, the processor 102 converts the film formation-related material name included in one extraction target sentence into a uniform material name such as an IUPAC name (step S107).

Next, the processor 102 edits the intermediate file (step S108). More specifically, the processor 102 adds one row to the intermediate file and writes a material number corresponding to the material in the M column as illustrated in FIG. 6. Further, in C columns of the intermediate file representing the compositions of the material, the processor 102 sets a value of each column (“C1 to C5” in FIG. 6) corresponding to the name of a functional group, a metal, or the like represented by the IUPAC name or the like to 1 and sets a value of the other column to 0. The data related to the functional group, the metal, or the like represented by the IUPAC name is stored in the auxiliary storage device 105 in advance.

When a plurality of materials are included in one sentence, the processor 102 adds a row for each of the materials and edits the intermediate file. For example, the second and third rows of the intermediate file illustrates in FIG. 6 have the same value “1” in the P column but have the values “1” and “2” in the M column. This indicates that two materials are included in one sentence.

[our] After the processor 102 repeatedly performs the processing in step S107 and S108 until the end of one sentence (step S109), the processing proceeds to step Silo via connector A, and the processing from step S104 to step S109 is further performed until the end of the paragraph of “experimental method” included in the paper data (step Silo).

Next, the processor 102 searches a specified paragraph such as the paragraph of “summary” in the paper data, from which the material names have been extracted, for a function name corresponding to a search condition, and if the matching function name is found (step S112: YES), the processor 102 edits the intermediate file (step S113).

More specifically, the processor 102 writes 1 in the F column indicating the function in the processing target paper data set having the same title. If no function name is hit in the search (step S112: NO), the value in the F column is set to 0. For example, as illustrated in FIG. 6, “1” is written as each of the values of the liquid repellency (F1) and transparency (F3), corresponding to the paper data set having the same title, which is indicated by the values “1” in the T column from the first row to the fifth row of the intermediate file.

Next, the processor 102 executes searches for all of the plurality of preset function names (step S114). Further, when the above processing has been performed on all the paper data sets stored in the document DB 10 (step S115), the processor 102 closes the intermediate file (step S116).

[Learning Data Generation Processing]

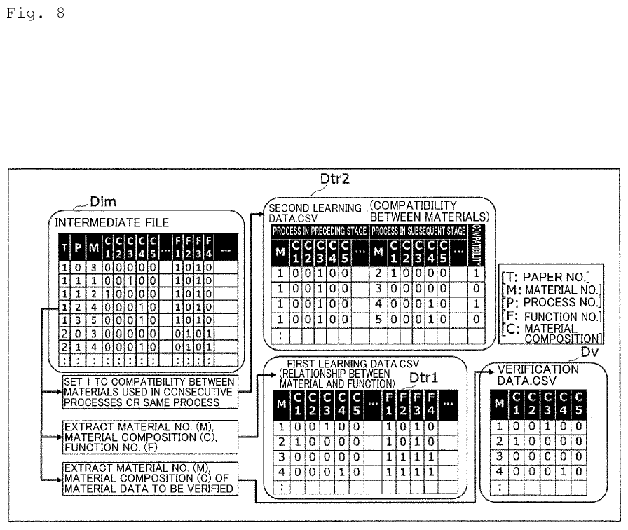

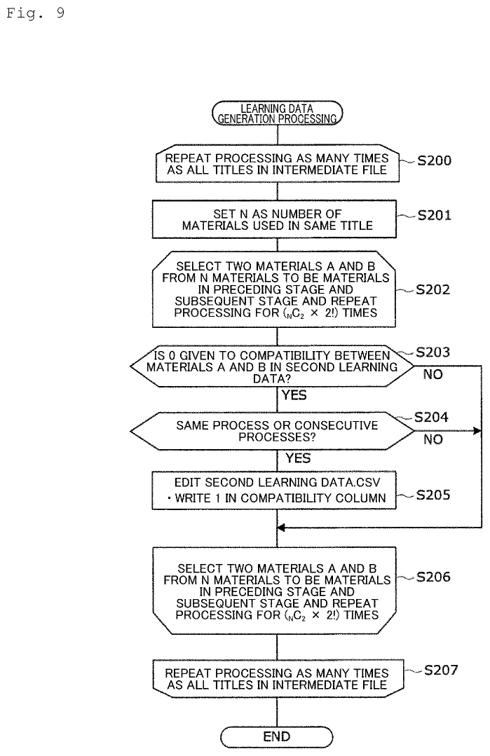

Next, a specific example of learning data generation processing by the learning data generation unit 13 implemented by the processor 102 will be described with reference to FIGS. 8 and 9.

As illustrated in FIG. 8, the learning data generation unit 13 generates first learning data (“Dtr1” in FIG. 8) and second learning data (“Dtr2” in FIG. 8) based on the intermediate file created from the film formation-related “materials” and “functions” extracted by the first extraction unit 11 and the second extraction unit 12. As with the intermediate file, data in CSV format can be used as these learning data.

The first learning data is learning data in which the materials and the functions are stored in association with each other. The learning data generation unit 13 extracts the material number (M), the material composition (C), and the function (F) stored in the intermediate file to generate the first learning data.

As the data structure of the second learning data, a material number (M) and material composition (C) of two materials and compatibility are set. The “compatibility” is defined as 1 for two materials used in the consecutive processes or the same process and 0 for the other cases. The “compatibility” reflects, for example, the properties of the material to be considered during the film formation.

Specific examples are as follows: i) a film of a negatively charged material can be formed on a positively charged surface so that this combination is likely to be used consecutively, whereas, a film of a positively charged material is difficult to be formed on a positively charged surface so that this combination is rarely used consecutively; ii) in addition, a hydrophobic material is easily adopted to a hydrophobic surface due to hydrophobic group-hydrophobic group interaction so that this combination is likely to be used simultaneously; iii) a material having a thiol group and a material having a vinyl group are likely to be used consecutively due to thiol-ene reaction. The compatibility between the two materials reflects a certain ordering applied when such a film-forming material is selected.

Next, the generation processing of the second learning data illustrated in FIG. 8 will be described with reference to a flowchart in FIG. 9.

As illustrated in FIG. 9, the processor 102 repeatedly performs processing from step S201 to step S206 as many times as the number of titles of the paper data sets stored in the intermediate file (step S200). More specifically, the processor 102 counts the number N of the materials used under the same title (the same value in the T column) in the intermediate file (step S201).

Next, the processor 102 randomly selects two materials from the N materials and repeats processing in which one of the materials is set as a material A in a preceding stage and the other is set as a material B in a subsequent stage for (NC2×2!) times (step S202). The processor 102 generates second learning data illustrated in FIG. 8. In the second learning data, “process in the preceding stage”, “process in the subsequent stage”, and “compatibility” are recorded in association with each other. In addition, a value “1” indicating good compatibility or a value “0” indicating poor compatibility is stored in advance in the “compatibility” column.

Next, if the value of the compatibility between the material A in the preceding stage and the material B in the subsequent stage selected in step S202 is 0 in the second learning data (step S203: YES), the processor 102 determines whether the material A in the preceding stage and the material B in the subsequent stage are used in the same process or the consecutive processes based on the values in the P column of the intermediate file (step S204). If the material A and the material B have the P-column values indicating the same process or the consecutive processes (step S204: YES), the value of the “compatibility” of the corresponding row and column in the second learning data is changed to “1” (step S205).

In contrast, if the compatibility between the material A and the material B is 1 in the second learning data (step S203: NO), the processing proceeds to step S206. In addition, in step S204, if the material A in the preceding stage and the material B in the subsequent stage are not in the same process or consecutive processes in the intermediate file (step S204: NO), the processing also proceeds to step S206. That is, the processor 102 does not change the value of the compatibility between the material A in the preceding stage and the material B in the subsequent stage in the second learning data.

Next, the processor 102 repeatedly performs the processing from step S203 to step S205 on the N materials for (NC2×2!) times, which is the total number of combinations (step S206). Further, after the values of the compatibility between the two materials have been updated for all the title numbers (numbers “1, 2, . . . ” in the T column) of the paper data sets in the intermediate file (step S207), the processing ends.

Learning Processing

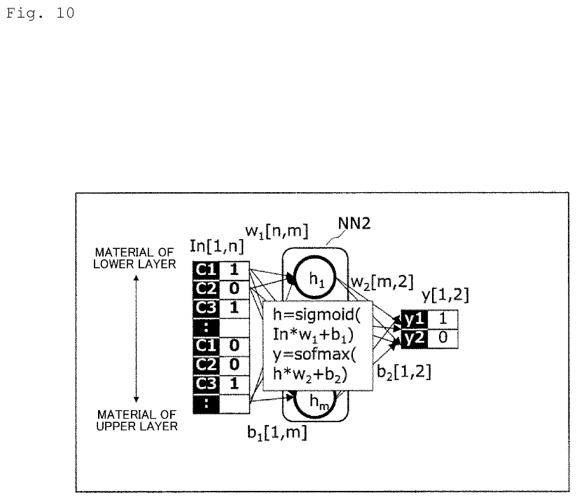

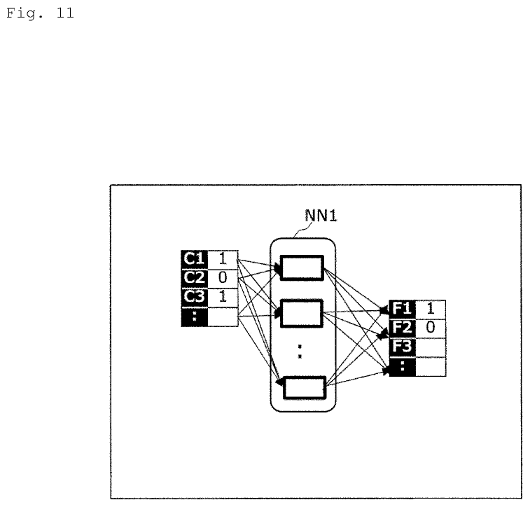

Next, learning processing performed by the learning processing unit 14 will be described with reference to FIGS. 10 and 11. FIG. 10 is a diagram illustrating the learning processing performed based on the second learning data.

The learning processing unit 14 trains a neural network NN2 by using the second learning data. As described above, the second learning data is data in which two materials and the compatibility between these two materials are associated with each other. In an example in FIG. 10, information about the material composition (C) used in the process in the preceding stage is illustrated on the input-In side, and the compatibility data is illustrated on the output-y side. In addition, in the examples in FIGS. 10 and 11, as the material composition (C), the material composition on the upper side of FIG. 10 indicates the material composition on the lower layer side of a multi-layer film, and the material composition on the lower side of FIG. 10 indicates the material composition on the upper layer side of the multi-layer film.

The learning processing unit 14 performs an operation of the neural network NN2 based on the material composition in the preceding stage given as an input, and adjusts, updates, and determines values of parameters such as weights so that the compatibility, which is a correct answer label, is output. In this way, the trained second learning model is obtained. The trained second learning model is a model in which the compatibility between the two materials in terms of a film-forming process is learned. The data structure of the input and output of the neural network NN2 is not limited to the example in FIG. 10.

As illustrated in FIG. 11, the learning processing unit 14 trains a neural network NM prepared in advance by using the first learning data. As described above, the first learning data is learning data indicating the relationship between the material and the function.

The learning processing unit 14 performs an operation of the neural network NM based on the material composition (C) given as an input, and adjusts and determines parameters such as weights so that the function (F), which is a correct answer label, is output. In this way, the trained first learning model is obtained. The first learning model is a model in which the function corresponding to the material is learned. The data structure of the input and output of the neural network NM is not limited to the example in FIG. 11. In addition, the example in FIG. 11 illustrates the case where the neural network NM has one correct answer label for the input. However, the learning may be performed for each function, and the neural network NM may have a plurality of correct answer labels for the input.

As described above, the materials development support apparatus 1 according to the first embodiment extracts preset words indicating a film formation-related “material” and a “function” of the “material” from a large number of paper data sets related to film formation and generates extraction data. Further, the materials development support apparatus 1 generates second learning data indicating the compatibility between the two materials in terms of the film forming process based on the extraction data. Further, the materials development support apparatus 1 generates first learning data indicating the function corresponding to the material based on the extraction data.

Further, the materials development support apparatus 1 trains a machine learning model prepared in advance by using the first learning data to obtain a trained first learning model in which the function corresponding to the material is learned.

The materials development support apparatus 1 trains a machine learning model prepared in advance by using the second learning data to obtain a trained second learning model in which the compatibility between the two materials in terms of the film forming process is learned.

As described above, the materials development support apparatus 1 more effectively collects information about the film formation from a large amount of text data and learns the compatibility between the materials and the function corresponding to the material. Thus, the materials development support apparatus 1 can support the user to develop the film formation materials.

In addition, the materials development support apparatus 1 learns the feature amount of the function with relatively low mathematical relevance, such as transparency, liquid repellency, and conductivity, as the function corresponding to the material. Thus, the materials development support apparatus 1 can support the user to develop the film forming materials more effectively.

Further, the materials development support apparatus 1 generates the learning data from “experimental method”, “summary”, and the like included in paper data so that the materials development support apparatus 1 can easily generate the learning data.

### Second Embodiment

Next, a second embodiment of the present invention will be described. In the following description, the same components as those in the first embodiment described above will be denoted by the same reference characters, and description thereof will be omitted.

In the first embodiment, the learning processing in which the first learning model in which the compatibility between materials related to film formation is learned and the second learning model in which a function corresponding to a material is learned are acquired by training the machine learning models prepared in advance has been described. In the second embodiment, inference processing is performed by using the first learning model and the second learning model that have been obtained by the learning processing.

In the inference processing performed by a materials development support apparatus 1A according to the present embodiment, as illustrated in FIG. 4, for example, a material of a substrate used when a multi-layer film is formed and functions requested for the multi-layer film are given as inputs, operations using the trained first learning model and the trained second learning model are performed, and a candidate for the structure of the multi-layer film is output. The candidate for the structure of the multi-layer film includes the film-forming materials in the vertical direction from the substrate, which are deemed to have the input functions.

In this respect, in a conventional method for acquiring a design guideline for the multi-layer film mainly by experiment, as illustrated in FIG. 4, first, the multi-layer film is designed, and based on the design, a thin film is formed, with the aim of achieving the exhibition of the desired function. The solving method according to this conventional example is called a solution of a forward problem. Whereas the materials development support apparatus 1A according to the present embodiment applies a method of solving an inverse problem in which the design of the multi-layer film is obtained from the functions, which is an opposite approach to that to the forward problem.

Functional Block of Materials Development Support Apparatus

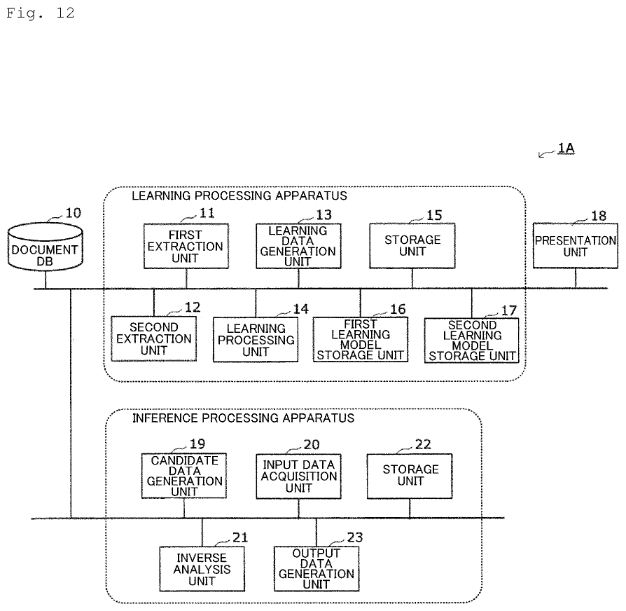

FIG. 12 is a block diagram illustrating a configuration of the materials development support apparatus 1A according to the present embodiment.

In addition to the functional units constituting the learning processing apparatus described in the first embodiment, the materials development support apparatus 1A includes a candidate data generation unit 19, an input data acquisition unit 20, an inverse analysis unit 21, a storage unit 22, and an output data generation unit 23 that constitute an inference processing apparatus. Hereinafter, a configuration different from that of the first embodiment will be mainly described.

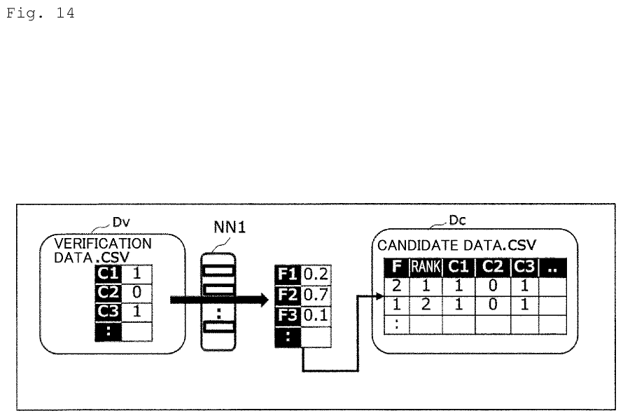

The candidate data generation unit 19 inputs verification data including a preset verification target material to the trained first learning model, performs an operation of the first learning model, checks the function of each material, outputs a plurality of candidates for the function provided by the verification target material, and generates candidate data (“Dc” in FIG. 14). As illustrated in FIG. 8, the verification data (Dv) is data obtained by extracting the material composition (C) of the material (M) to be verified, from the extraction data (intermediate file) of the “materials” and the “functions” extracted from the document data by the first extraction unit 11 and the second extraction unit 12.

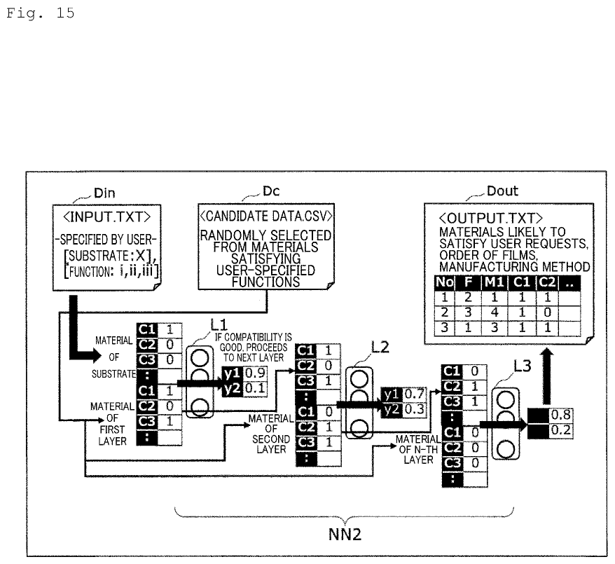

The input data acquisition unit 20 is data including information about a material of a substrate specified by the user and desired functions of the thin film that are received by the input device 107. The acquired input data (“Di” in FIG. 15) is stored in the storage unit 22.

The inverse analysis unit 21 provides the input data and data of the material randomly selected from the candidate data as inputs to the second learning model, performs an operation of the second learning model, and outputs the materials that are likely to satisfy the user request, the order of layers, and a manufacturing method as outputs.

The storage unit 22 stores the candidate data generated by the candidate data generation unit 19. The storage unit 22 also stores the output by the inverse analysis unit 21.

The output data generation unit 23 generates data indicating the candidate for the structure of the multi-layer film output from the inverse analysis unit 21.

The presentation unit 18 can display the output data (“Dout” in FIG. 15) on a display screen.

Inference Processing

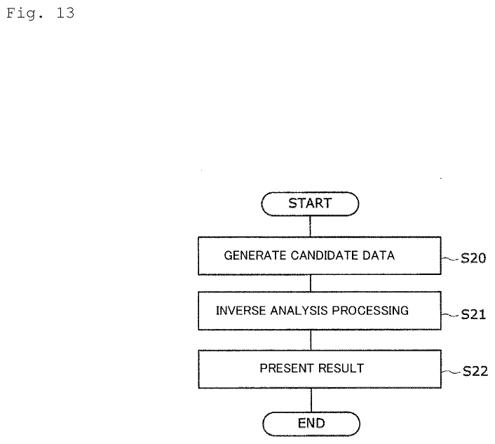

Next, inference processing performed by the materials development support apparatus 1A having the above-described functional configuration will be described with reference to a flowchart in FIG. 13. In the following description, it is assumed that the first learning model and the second learning model have previously been constructed by the learning processing performed by the learning processing apparatus illustrated in FIG. 12 and are stored in the first learning model storage unit 16 and the second learning model storage unit 17, respectively. It is also assumed that the verification data (FIG. 8) previously obtained by extracting the verification target material (M) and the material composition (C) from the extraction data (intermediate file) generated from the data extracted by the first extraction unit ii and the second extraction unit 12 is stored in the storage unit 22.

As illustrated in FIG. 13, first the candidate data generation unit 19 reads the trained first learning model from the first learning model storage unit 16, provides the verification data prepared in advance as an input, performs the operation of the first learning model, and outputs candidate data indicating candidates for the function of each material (step S20).

The candidate data is stored in the storage unit 22.

In addition, a material that is not stored in the intermediate file which is the extraction data, that is, a material that is not included in the paper data may be added to the verification data, and candidates for the function of such a material may be output in the candidate data. This may allow a completely new film to be presented as a candidate for the material development. The present embodiment makes it possible to present such a new film candidate since the material related to the film formation is grasped from various aspects, for example, by the functional group or the like.

FIG. 14 is a block diagram for describing an operation performed by the candidate data generation unit 19. In an example in FIG. 14, the neural network NN1 having one correct answer label in which the sum of the probabilities of the output results (F1, F2, F3, . . . ) is 1 is used. This indicates that the closer the relationship between the material and the function is, the closer to 1 the output value of the neural network NN1 becomes. Thus, the candidate data includes information about the ranks of the functions included in the input data. The material corresponding to the input is likely to have a higher-ranking function, and the material having the lower-ranking function is less likely to have the function included in the input data.

As described above, by generating the candidate data by using the trained first learning model, the material that is relatively less likely to satisfy the function specified in the input data acquired by the input data acquisition unit 20 can be eliminated in advance. Of course, a single material can have a plurality of functions, and if so, a machine learning algorithm that calculates the probability of each function can be used. In that case, since the probabilities are presented per function, determination processing can be performed by using a predetermined threshold. In this way, the candidate data generation unit 19 obtains candidate data, which are items of the materials corresponding to the function, by performing the operation of the trained first learning model.

Returning to FIG. 13, the inverse analysis unit 21 provides the input data acquired by the input data acquisition unit 20 and the candidate data as inputs, performs an operation of the trained second learning model, and performs inverse analysis processing for outputting a candidate for the structure of the multi-layer film (step S21). The output data generation unit 23 generates output data indicating candidates for the materials of the multi-layer film and the order of the films based on the output from the inverse analysis unit 21.

Next, the presentation unit 18 displays the output data generated by the output data generation unit 23 on the display screen (step S22).

Inverse Analysis Processing

First, an outline of inverse analysis processing will be described with reference to FIG. 15.

As illustrated in FIG. 15, the input data and random data included in the candidate data are provided as inputs to the trained second learning model. The input data includes the material of a substrate specified by the user and the functions requested for the multi-layer film. As the input data, for example, data in text format can be used.

The random data selected from the candidate data includes the material randomly selected from the materials satisfying the functions specified by the user in the input data and is input to the trained second learning model as the material to serve as the first layer constituting the multi-layer film.

The neural network NN2 illustrated in FIG. 15 is the trained second learning model, and FIG. 15 illustrates the input and output of a first layer L1, a second layer L2, and a third layer L3 of the neural network NN2.

As described above, the substrate material specified by the user is input from the input data to the first layer L1 of the neural network NN2, and the material selected from the materials satisfying the functions specified by the user is input from the candidate data to the first layer L1 of the neural network NN2 as the material of the first layer of the multi-layer film. The neural network NN2 is a learning model that has learned the compatibility between the materials and outputs the compatibility between the input material of the substrate and the input material of the first layer of the multi-layer film by performing an operation of the neural network NN2.

When the inference result indicating that the input material of the substrate has good compatibility with the input material of the first layer of the multi-layer film is obtained from the output of the first layer L1 of the neural network NN2, an operation of the second layer L2 of the neural network NN2 is performed. In the second layer L2, the material of the first layer of the multi-layer film, which has good compatibility with the substrate material, and the material randomly selected from the materials satisfying the functions specified by the user in the candidate data to serve as the material of the second layer of the multi-layer film are provided as inputs. Likewise, the compatibility between the material of the first layer and the material of the second layer of the multi-layer film is output as the operation result of the neural network NN2, and if the output indicating that the compatibility between these materials is good is obtained, the operation of the neural network NN2 is repeatedly performed on each of the materials from the third layer until the N-th layer of the multi-layer film.

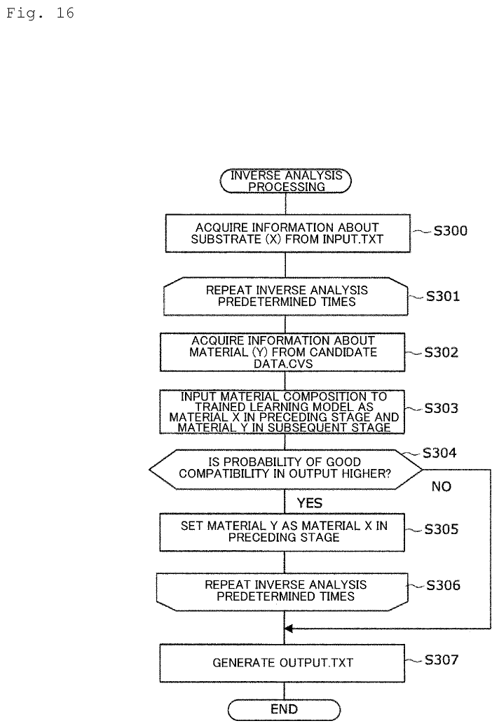

Next, the inverse analysis processing by the inverse analysis unit 21 implemented by the processor 102 will be described with reference to a flowchart in FIG. 16.

First, the processor 102 acquires information indicating a material X of the substrate specified by the user from the input data (step S300). Next, the processor 102 repeatedly performs the inverse analysis processing from step S302 to step S305 a predetermined number of times (step S301). More specifically, the processor 102 acquires information indicating, for example, a material Y of the multi-layer film from the candidate data (step S302).

Next, the processor 102 provides the material X as the preceding stage process and the material Y as the subsequent stage process as inputs to the second learning model that has previously learned the material composition (C) of each material (step S303).

Next, the processor 102 performs an operation of the second learning model and obtains probability values for respective classes of “good compatibility” and “poor compatibility” between the material X and the material Y as outputs, and if the probability value of “good compatibility” is higher than the probability value of “poor compatibility” (step S304: YES), the processor 102 performs the operation of the second learning model by using the material Y in the subsequent stage as the material in the preceding stage (step S30₅).

Next, the processor 102 performs the inverse analysis processing a predetermined number of times (step S306), and then generates output data (step S307). In contrast, in step S304, if the probability value of “poor compatibility” between the two materials is higher (step S304: NO), the processing proceeds to steps S307, and the processor 102 generates output data (step S307).

By performing the above processing, sequential candidates for the materials in the vertical direction from the substrate can be obtained as output data. In the example of the inverse analysis processing described with reference to FIG. 16, in step S304, if the compatibility between the materials is determined to be poor, the processing ends. However, the operation of the neural network NN2 of the subsequent stage may be continued. Further, in step S302, if a specific material, for example, a material that frequently appears on the outermost surface is selected, the processing may be arranged to end (step S307).

Further, in view of the temperature at the time of film formation, the solubility in the solvent, or the like, by giving constraints to the material selected from the candidate data in step S302, the materials to be input as candidates may be narrowed down in advance. The constraints are previously stored in the storage unit 22.

In addition to the above constraints, for example, the film thickness, roughness of the surface, porosity, etc. are also important factors for allowing the multi-layer film to exhibit the specified functions. Therefore, such information can be arranged to be taken into consideration upon selecting the material from the candidate data.

Specific Example of Configuration of Materials Development Support Apparatus

An example of a specific configuration of the materials development support apparatus 1A having the above-described configuration will be described with reference to the block diagram in FIG. 3. For example, the materials development support apparatus 1A can be implemented by the servers 100, 200, and the communication terminal device 300. The servers 100, 200, and the communication terminal device 300 are connected via the communication network NW. A flow indicated by a solid line in FIG. 3 is a processing flow performed by the learning processing apparatus included in the materials development support apparatus 1A according to the present embodiment (“learning phase” in FIG. 3).

In addition, a flow indicated by a dashed line in FIG. 3 is a processing flow performed by the inference processing apparatus included in the materials development support apparatus 1A according to the present embodiment (“inference phase” in FIG. 3). Thus, the learning processing apparatus of the materials development support apparatus 1A according to the present embodiment is implemented by the servers 100 and 200, and the inference processing apparatus is implemented by the server 200 and the communication terminal device 300.

The server 100 includes, for example, the document DB 10, the first extraction unit 11, the second extraction unit 12, and the learning data generation unit 13 described with reference to FIG. 12.

The server 200 includes, for example, the learning processing unit 14, the first learning model storage unit 16, the second learning model storage unit 17, the candidate data generation unit 19, the storage unit 22, and the inverse analysis unit 21 described with reference to FIG. 12.

The servers 100, 200, and the communication terminal device 300 are implemented by a computer configuration including the processor, the main storage device, the communication I/F, and the auxiliary storage device described with reference to FIG. 2. Further, as illustrated in FIG. 3, the server 100 transmits the generated learning data to the server 200 via the communication network NW. The communication terminal device 300 and the server 200 exchange data via the communication network NW.

As described above, the materials development support apparatus 1A according to the present embodiment can be implemented by the configuration in which each function illustrated in FIG. 1 is distributed on the network.

Effects of Materials Development Support Apparatus

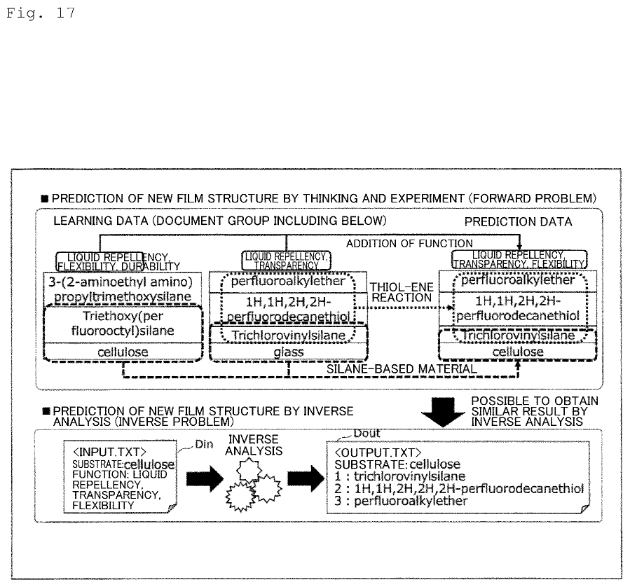

Next, effects of the materials development support apparatus 1A according to the present embodiment will be described with reference to FIG. 17.

FIG. 17 is a diagram for describing the results of the learning processing and the inference processing performed by the materials development support apparatus 1A according to the present embodiment. In the present example, by using learning data related to film formation extracted from 39 papers (“Avijit Baidya, ACS Nano 2017, 11, 11091-11099”, “Junsheng Li, Nano Lett. 2015, 15, 675-681.”, etc.) randomly selected from the literature on laminated thin films of organic, inorganic, and metallic materials, whether or not a film forming method included in one paper (“Jiaqi Guo, ACS Appl. Mater. Interfaces 2016, 8, 34115-34122.”) not used for the learning can be predicted has been verified.

The upper portion of FIG. 17 illustrates a procedure for predicting the structure of a film as a forward problem solved by orientation and experiments, which is a conventional example. The lower portion of FIG. 17 illustrates solving processing as an inverse problem in which the materials development support apparatus 1A according to the present embodiment obtains the structure of a film as an output by inputting the material of a substrate and the requested functions to the trained machine learning model.

In the lower portion of FIG. 17, “cellulose” is specified as the material of a substrate, and “liquid repellency”, “transparency”, and “flexibility” are specified as the functions in input data (“input.txt”). That is, a method for producing “transparent, flexible, and stain-free paper” is tried to be obtained by the inverse analysis.

Further, as a result of the inverse analysis, output data (“output.txt”) suggesting that a film be formed with “trichlorovinylsilane”, “1H, 1H, 2H, 2H-perfluorodecanethiol”, and “perfluoroalkylether” in the vertical direction from the substrate can be obtained. This is a material selection result close to the manufacturing method in the one paper not used for the learning. Therefore, it can be said that this is a highly feasible solution.

In contrast, in the conventional example illustrated in the upper portion of FIG. 17, an experimental method for searching for the material that exhibits a new function is performed while referring to a large number of paper data sets. In this case, too, by trying combining the materials used in the data being learned and the function of the film created by these materials, it can be inferred that there is relevance between the manufacturing method of the film in the paper not used for the learning and the achieved function.

In other words, it can be said that the materials development support apparatus 1A according to the present embodiment is a technique that imitates one of the thinking methods that a human uses to develop a new technique by means of the inverse analysis using machine learning. Furthermore, not only imitating but also more rational material selection without depending on subjectivity or detection of the user can be achieved, and a comprehensive search can be performed even on a volume of the material combinations that is deemed to be impossible to handle manually.

As described above, according to the second embodiment, since the inverse analysis processing is performed by using the trained first learning model and the trained second learning model, a candidate for the design of a multi-layer film having a plurality of functions can be more easily presented.

In the embodiment described above, the case where the materials development support apparatus 1A includes the learning processing apparatus and the inference processing apparatus has been described with reference to FIG. 12. However, the inference processing apparatus may be configured independently from the learning processing apparatus.

While the embodiments of the materials development support apparatus, the materials development support method, and the materials development support program according to embodiments of the present invention have thus been described, the present invention is not limited to the embodiments described above, and various modifications conceivable by those skilled in the art can be made within the scope of the invention recited in the claims. For example, the order of each step in the materials development support method is not limited to that described above.

### REFERENCE SIGNS LIST

1, 1A Materials development support apparatus

10 Document DB

11 First extraction unit

12 Second extraction unit

13 Learning data generation unit

14 Learning processing unit

15, 22 Storage unit

16 First learning model storage unit

17 Second learning model storage unit

18 Presentation unit

19 Candidate data generation unit

20 Input data acquisition unit

21 Inverse analysis unit

23 Output data generation unit

100, 200 Server

300 Communication terminal device

101 Bus

102 Processor

103 Main storage device

104 Communication I/F

105 Auxiliary storage device

106 Input-output I/O

107 Input device

108 Display device

1-7. (canceled)

8. A materials development support apparatus comprising:
an input data acquisition device configured to acquire input data including a material of a base forming a thin film and a function of the thin film;
a candidate data generator configured to provide a preset verification target material as an input to a first learning model in which a relationship between an individual one of a plurality of materials used for forming a thin film and a function provided by the material is previously learned, perform an operation of the first learning model, output a plurality of candidates for a function provided by the verification target material, and generate candidate data;
an inverse analyzer configured to select a material that provides the function of the thin film included in the input data from the plurality of candidates for the function included in the candidate data, provide the material of the base included in the input data and the selected material as inputs to a second learning model in which compatibility with the base forming the thin film is previously acquired by learning, perform an operation of the second learning model, and output a candidate for structure of the thin film; and
a presenter configured to present the candidate for the structure of the thin film output by the inverse analyzer

9. The materials development support apparatus according to claim 8, further comprising:
a first extractor configured to extract a plurality of preset function names indicating the function of the thin film from an individual one of a plurality of document data; and
a second extractor configured to extract a plurality of preset material names indicating the material used for forming the thin film from an individual one of a plurality of document data.

10. The materials development support apparatus according to claim 9, further comprising:
a first learning data generator configured to generate first learning data in which a material and a function provided by the material are associated with each other for each of the plurality of material names, based on the plurality of function names extracted by the first extractor and the plurality of material names extracted by the second extractor; and
a second learning data generator configured to generate second learning data in which the individual material indicated by the plurality of material names and compatibility with the base forming the thin film are associated with each other, based on the plurality of function names extracted by the first extractor, the plurality of material names extracted by the second extractor, and extraction-source document data.

11. The materials development support apparatus according to claim 10, further comprising:
a first learning processor configured to train a preset first machine learning model by using the first learning data and construct the first learning model in which a relationship between a material and a function provided by the material is learned;
a second learning processor configured to train a preset second machine learning model by using the second learning data and construct the second learning model in which compatibility with the base forming the thin film is acquired by learning;
a first learning model storage device configured to store the trained first learning model; and
a second learning model storage device configured to store the trained second learning model.

12. A materials development support method comprising:
acquiring input data including a material of a base forming a thin film and a function of the thin film;
providing a preset verification target material as an input to a first learning model in which a relationship between an individual one of a plurality of materials used for forming a thin film and a function provided by the material is previously learned;
performing an operation of the first learning model;
outputting a plurality of candidates for a function provided by the verification target material;
generating candidate data;
selecting a material configured to provide the function of the thin film included in the input data from the plurality of candidates for the function included in the candidate data;
providing the material of the base included in the input data and the selected material as inputs to a second learning model in which compatibility with the base forming the thin film is previously acquired by learning;
performing an operation of the second learning model;
outputting a candidate for structure of the thin film; and
presenting the candidate for the structure of the thin film output.

13. The materials development support method according to claim 12, comprising:
extracting a plurality of preset function names indicating the function of the thin film from an individual one of a plurality of document data; and
extracting a plurality of preset material names indicating the material used for forming the thin film from an individual one of a plurality of document data.

14. The materials development support method according to claim 13, comprising:
generating first learning data in which a material and a function provided by the material are associated with each other for each of the plurality of material names, based on the plurality of function names extracted in the first extraction process and the plurality of material names extracted in the second extraction process; and
generating second learning data in which the individual material indicated by the plurality of material names and compatibility with the base forming the thin film are associated with each other, based on the plurality of function names extracted in the first extraction process, the plurality of material names extracted in the second extraction process, and the extraction-source document data.

15. The materials development support method according to claim 14, comprising:
training a preset first machine learning model by using the first learning data and constructs the first learning model in which a relationship between a material and a function provided by the material is learned;
training a preset second machine learning model by using the second learning data and constructs the second learning model in which compatibility with the base forming the thin film is acquired by learning;
storing the trained first learning model in a first learning model storage device; and
storing the trained second learning model in a second learning model storage device.

16. A materials development support program that causes a computer to execute:
an input data acquisition process that acquires input data including a material of a base forming a thin film and a function of the thin film;
a candidate data generation process that provides a preset verification target material as an input to a first learning model in which a relationship between an individual one of a plurality of materials used for forming a thin film and a function provided by the material is previously learned, performs an operation of the first learning model, outputs a plurality of candidates for a function provided by the verification target material, and generates candidate data;
an inverse analysis process that selects a material that provides the function of the thin film included in the input data from the plurality of candidates for the function included in the candidate data, provides the material of the base included in the input data and the selected material as inputs to a second learning model in which compatibility with the base forming the thin film is previously acquired by learning, performs an operation of the second learning model, and outputs a candidate for structure of the thin film; and
a presentation process that presents the candidate for the structure of the thin film output in the inverse analysis process.

17. The materials development support program according to claim 16 that causes the computer to further execute:
a first extraction process that extracts a plurality of preset function names indicating the function of the thin film from an individual one of a plurality of document data; and
a second extraction process that extracts a plurality of preset material names indicating the material used for forming the thin film from an individual one of a plurality of document data;

18. The materials development support program according to claim 17 that causes the computer to further execute:
a first learning data generation process that generates first learning data in which a material and a function provided by the material are associated with each other for each of the plurality of material names, based on the plurality of function names extracted in the first extraction process and the plurality of material names extracted in the second extraction process; and
a second learning data generation process that generates second learning data in which the individual material indicated by the plurality of material names and compatibility with the base forming the thin film are associated with each other, based on the plurality of function names extracted in the first extraction process, the plurality of material names extracted in the second extraction process, and the extraction-source document data.

19. The materials development support program according to claim 18 that causes the computer to further execute:
a first learning processing process that trains a preset first machine learning model by using the first learning data and constructs the first learning model in which a relationship between a material and a function provided by the material is learned; and
a second learning processing process that trains a preset second machine learning model by using the second learning data and constructs the second learning model in which compatibility with the base forming the thin film is acquired by learning.

20. The materials development support program according to claim 19 that causes the computer to further execute:
a first learning model storage process that stores the trained first learning model in a first learning model storage device; and
a second learning model storage process that stores the trained second learning model in a second learning model storage device.
